(19) United States
(12) Patent Application Publication
(10) Pub. No.: US 2011/0127188 A1
(43) Pub. Date: Jun. 2, 2011

(54) Method of Using Coextruded Film for Sterile Barrier System to Deliver Seal and Peel Characteristics

(75) Inventors: Patrick W. Thompson, Greenville, SC (US); Jacqueline W. Smith, Moore, SC (US)

(73) Assignee: Cryovac, Inc., Duncan, SC (US)

(21) Appl. No.: 12/956,197

(22) Filed: Nov. 30, 2010

Related U.S. Application Data
(60) Provisional application No. 61/283,222, filed on Dec. 1, 2009.

(51) Int. Cl. A61B 19/02; B65D 85/00; B65B 43/26; B32B 27/08; D04H 13/00

(52) U.S. Cl. 206/438; 206/524.6; 53/492; 428/516; 442/327

(57) ABSTRACT

The presently disclosed subject matter is directed to a multilayer coextruded film that can be used in combination with uncoated medical grade paper or uncoated Tyvek® to produce a sterile barrier system that delivers desired seal and peel characteristics with no paper tear. The combination can be particularly suitable for medical device packaging where the package is subjected to gas sterilization, such as with ethylene oxide.

### FIELD OF THE INVENTION

The presently disclosed subject matter relates generally to packaging and packaging containers suitable for packaging a wide variety of medical supplies and devices. More particularly, the presently disclosed subject matter relates to packages that are sterilizable and readily openable after sterilization with minimal tearing and/or particulate generation accumulation.

### BACKGROUND

Many products, especially devices and supplies used in surgical and other medical applications, must be sterilized prior to use. Examples of such products in the medical context include (but are not limited to) surgical devices, tubing, valves, gauzing, syringes, and protective clothing such as surgical gowns and gloves. Such products and supplies are often packaged in breather packages prior to being sterilized.

Breather packages are containers in which a sheet of clear impermeable plastic is adhered to a semi-permeable paper, cardboard, or other fibrous material backing to provide a sterile display environment for the medical product. Such breather pouches allow sterilizing gases, such as ethylene oxide, to pass through the semi-permeable backing to effect sterilization of the medical product and also prevent microorganisms from entering the pouch after the sterilization process is complete. Examples of such packages are widely known and include, but are not limited to, U.S. Pat. Nos. 3,991,881 to Augurt; 4,183,431 to Schmidt et al.; 5,217,772 to Brown et al.; and 5,418,022 to Anderson et al., the entire disclosures of which are incorporated by reference herein.

However, several distinct problems can occur upon opening breather packages of the prior art, particularly within the sterile environment of an operating room. For example, when the impermeable plastic layer is adhered to the fibrous backing layer, whether by means of adhesives, heat seals or any other method, there is a tendency for the fibrous layer to separate from itself so that the fibers pull apart from each other. This occurrence (known as “fiber-pull” or “fiber tear”) can be problematic in operating rooms and/or other medical settings. Particularly, the presence of a large number of particulate fibers is a detriment to the sterile conditions required in the operating room and other medical environments.

In addition, prior art breather packages have fibrous layers that can delaminate instead of easily separating from the plastic layer, allowing the medical device to remain encapsulated in the breather pouch. The surgical team must then make a further attempt to open the package which can result in a critical delay potentially affecting the health of the patient. Furthermore, to open such a package, a sharp instrument may be necessary to puncture the pouch, thereby compromising the sterility of the instrument and packaged product.

In order to overcome the fiber-pull and opening problems, the entire surface of the fibrous layer is commonly coated with a layer of plastic (called a “release agent”). However, the application of the release agent results in increased cost required to produce the breather package as an extensive amount of release agent material is used to cover the entire surface of the fibrous layer. In addition, after the pouch is sealed, the pouch is subjected to a sterilizing gas which passes through the fibrous layer. In the prior art, the pouches having adhesive material coating the entire surface of the fibrous layer suffer decreased porosity, and accordingly, require longer sterilization times.

The package disclosed herein obviates the disadvantages encountered in the prior art and provides a breather package that substantially reduces or eliminates fiber-pull associated with the prior art without unduly increasing the cost of the package. In addition, the disclosed package separates easily to reveal the product enclosed therein.

### SUMMARY

The presently disclosed subject matter is directed to a microbial resistant package comprising a gas-impermeable polymeric material comprising a cavity formed therein for receiving a product. In some embodiments, the polymeric material comprises a flange surrounding the cavity. An uncoated gas-permeable material is bonded to the flange of the polymeric material.

In some embodiments, the presently disclosed subject matter is directed to a method of eliminating fiber pull from a package. The method comprises providing a package comprising a gas-impermeable polymeric material comprising a cavity formed therein for receiving a product. The package also comprises an uncoated gas-permeable material bonded to the flange of the polymeric material. The method comprises opening the package by removing the uncoated gas permeable material to expose a product packaged therein.

In some embodiments, the presently disclosed subject matter is directed to a film suitable for use as a package in combination with an uncoated, permeable substrate. The film comprises a polyethylene sealant layer and at least one alternating interior layer comprising ethylene/vinyl acetate copolymer and polypropylene. The film is characterized by an easy-to-open and fiber-free peel.

### BRIEF DESCRIPTION OF THE DRAWINGS

FIG. 1a is a perspective view of one embodiment of the disclosed package.

FIG. 1b is a perspective view of the partially opened package of FIG. 1a.

FIG. 2a is a perspective view of one embodiment of a lower sheet used in accordance with the presently disclosed subject matter.

FIG. 2b is a perspective view of one embodiment of a lower sheet used in accordance with the presently disclosed subject matter.

FIG. 3a is a perspective view of one embodiment of a lidding sheet used in accordance with the presently disclosed subject matter.

FIG. 3b is a side elevation view of one embodiment of a lower sheet and a lidding sheet used in accordance with the presently disclosed subject matter.

FIG. 3c is a side elevation view of one embodiment of the disclosed package.

FIG. 4 is a side elevation view of one embodiment of an apparatus that can be used to make presently disclosed packages.

### DETAILED DESCRIPTION

### I. General Considerations

The presently disclosed subject matter is directed to a package comprising a multilayer coextruded film that can be used in combination with uncoated medical grade paper or uncoated spun bound polyolefin, such as Tyvek® to produce a sterile barrier system that delivers the desired seal and peel characteristics with no paper tear. The combination can be particularly suitable for medical device packaging where the package is subjected to gas sterilization, such as with ethylene oxide.

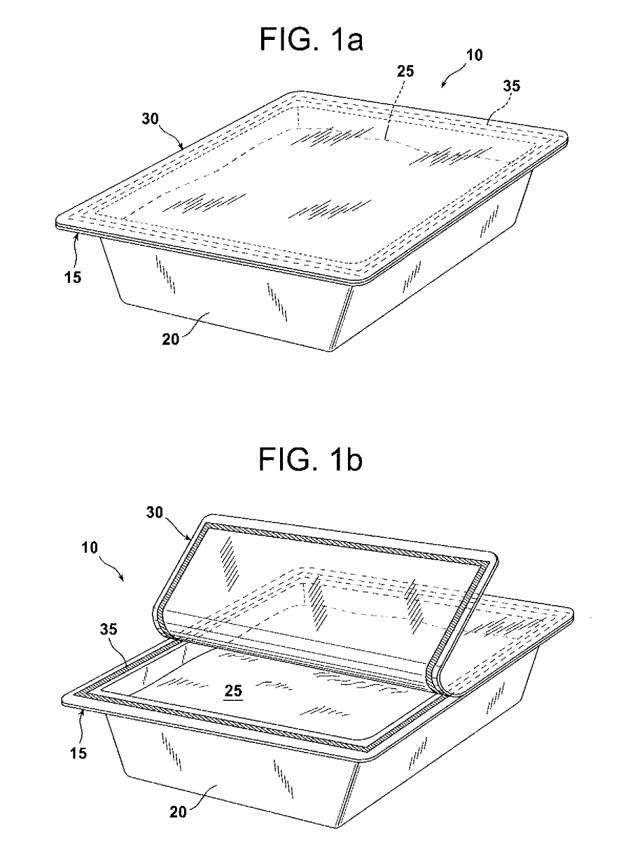

To elaborate, FIG. 1a illustrates one embodiment of a package of the presently disclosed subject matter. Package 10 comprises lower sheet 15 constructed of polymeric material, a portion of which has been formed into pocket 20. Product 25 (which can be a surgical drape, for example) is placed within pocket 20. Package 10 also comprises lidding sheet 30 of uncoated Tyvek® or uncoated medical grade paper. Peel seal area 35 surrounds pocket 20 where sheets 15 and 30 are joined. Package 10 is permeable to gases via lidding sheet 30 but is impervious to microorganisms. Thus, the package can be sterilized with gases such as ethylene oxide to create a sterile package.

As illustrated in FIG. 1b, in use lidding sheet 30 can simply be separated from lower sheet 15 at peel seal 35 by applying pressure to the lidding sheet. The construction of package 10 is such that the uncoated lidding sheet enables the package to have the desired seal strength while maintaining a clean peel opening free of fiber pull, rips, and tears. In addition, uncoated Tyvek® and uncoated medical grade paper are less costly compared to coated versions thereof.

### II. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long standing patent law convention, the terms “a”, “an”, and “the” refer to “one or more” when used in the subject application, including the claims. Thus, for example, reference to “a package” includes a plurality of such packages, and so forth.

Unless indicated otherwise, all numbers expressing quantities of components, reaction conditions, and so forth used in the specification and claims are to be understood as being modified in all instances by the term “about.” Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, percentage, and the like can encompass variations of, and in some embodiments, ±20%, in some embodiments ±10%, in some embodiments ±5%, in some embodiments ±1%, in some embodiments ±0.5%, and in some embodiments ±0.1%, from the specified amount, as such variations are appropriated in the disclosed package and methods.

As used herein, the terms “barrier” and “barrier layer” as applied to films and/or film layers, refer to the ability of a film or film layer to serve as a barrier to gases and/or odors. Examples of polymeric materials with low oxygen transmission rates useful in such a layer can include: ethylene/vinyl alcohol copolymer (EVOH), polyvinylidene dichloride (PVDC), vinylidene chloride copolymer such as vinylidene chloride/methyl acrylate copolymer, vinylidene chloride/vinyl chloride copolymer, polyamide, polyester, polyacrylonitrile (available as Barex™ resin), or blends thereof. Oxygen barrier materials can further comprise high aspect ratio fillers that create a tortuous path for permeation (e.g., nanocomposites). Oxygen barrier properties can be further enhanced by the incorporation of an oxygen scavenger, such as an organic oxygen scavenger. In some embodiments, metal foil, metallized substrates (e.g., metallized polyethylene terephthalate ((PET)), metallized polyamide, and/or metallized polypropylene), and/or coatings comprising SiOx or AlOx compounds can be used to provide low oxygen transmission to a package. In some embodiments, a barrier layer can have a gas (e.g., oxygen) permeability of less than or equal to about 500 cc/m²/24 hrs/atm at 73° F., in some embodiments less than about 100 cc/m²/24 hrs/atm at 73° F., in some embodiments less than about 50 cc/m²/24 hrs/atm at 73° F., and in some embodiments less than about 25 cc/m²/24 hrs/atm at 73° F.

The term “bulk layer” as used herein refers to a layer used to increase the abuse-resistance, toughness, modulus, etc., of a film. In some embodiments, the bulk layer can comprise polyolefin (including but not limited to) at least one member selected from the group comprising ethylene/alpha-olefin copolymer, ethylene/alpha-olefin copolymer plastomer, low density polyethylene, and/or linear low density polyethylene and polyethylene vinyl acetate copolymers.

The term “coated” refers to materials coated with a layer of plastic (the “release agent”). Similarly, the term “uncoated” refers to materials that are not coated with a release agent.

As used herein, the phrase “easy open” refers to any means for accessing the contents of a package that obviates the need to cut and/or pierce the package with a knife, scissors, or any other sharp implement. An easy open feature can be in at least one portion of the web used to form a package and can include one or more cuts, notches, or surface-roughened areas, lines of structural weakness, or combinations thereof. Examples of such easy open features are described in U.S. Patent Application Publication Nos. 2005/0084636 to Papenfuss et al. and 2005/0254731 to Berbert et al., both of which are incorporated herein in their entireties. In some embodiments, the easy open feature can include one or more frangible or peelable layers adapted to manually separate or delaminate at least a portion of the web used to form the package, as described in Reissued U.S. Pat. No. RE37,171 to Busche et al., which is incorporated herein in its entirety. It will be appreciated that in some embodiments peelable webs can further comprise one or more reclosable peelable layers. Examples of still other alternative easy open features include reclosable interlocking fasteners attached to at least a portion of the web used to form the package. Reclosable fasteners, in general, are known and are taught, for example, in U.S. Pat. Nos. 5,063,644; 5,301,394; 5,442,837; 5,964,532; 6,409,384; 6,439,770; 6,524,002; 6,527,444; 6,609,827; 6,616,333; 6,632,021; 6,663,283; 6,666,580; 6,679,027; and U.S. Patent Application Nos. 2002/0097923; and 2002/0196987, all hereby incorporated by reference in their entireties.

As used herein, the term “fiber pull” refers to

As used herein, the term “film” can be used in a generic sense to include plastic web, regardless of whether it is film or sheet.

As used herein, the term “impermeable” refers to a material that does not readily allow gas to pass through the material. In addition, the gas impermeable material also fails to allow airborne microbes, bacteria, viruses and mixtures thereof to pass through the material.

The term “package” as used herein refers to packaging materials configured around a product being packaged, and can include (but is not limited to) bags, pouches, and the like.

As used herein, the term “permeable” refers to a material that allows gas to pass through the material but fails to allow airborne microbes, bacteria, viruses and mixtures thereof to pass through the material. Gas permeable materials are also referred to in the art as “breathable materials.”

As used herein, the term “polymer” refers to the product of a polymerization reaction, and can be inclusive of homopolymers, copolymers, terpolymers, etc. In some embodiments, the layers of a film can consist essentially of a single polymer, or can have additional polymer together therewith, i.e., blended therewith.

As used herein, the term “seal” refers to any seal of a first region of an outer film surface to a second region of an outer film surface, including heat or any type of adhesive material, thermal or otherwise. In some embodiments, the seal can be formed by heating the regions to at least their respective seal initiation temperatures. The sealing can be performed by any one or more of a wide variety of means, including (but not limited to) using a heat seal technique (e.g., melt-bead sealing, thermal sealing, impulse sealing, dielectric sealing, radio frequency sealing, ultrasonic sealing, hot air, hot wire, infrared radiation).

As used herein, the phrases “seal layer”, “sealing layer”, “heat seal layer”, and “sealant layer”, refer to an outer film layer, or layers, involved in the sealing of the film to itself, another film layer of the same or another film, and/or another article that is not a film. It should also be recognized that in general, up to the outer 3 mils of a film can be involved in the sealing of the film to itself or another layer. In general, a sealant layer sealed by heat-sealing layer comprises any thermoplastic polymer. In some embodiments, the heat-sealing layer can comprise, for example, thermoplastic polyolefin, thermoplastic polyamide, thermoplastic polyester, and thermoplastic polyvinyl chloride. In some embodiments, the heat-sealing layer can comprise thermoplastic polyolefin.

As used herein, the term “sterilize” or “sterilization” refers to a wide variety of techniques employed to attenuate, kill or eliminate harmful or infectious agents. Examples of sterilization procedures include, for example, gas plasma sterilization, steam sterilization, ozone sterilization, hydrogen peroxide sterilization, ethylene oxide sterilization, and irradiation.

As used herein, the term “tie layer” refers to an internal film layer having the primary purpose of adhering two layers to one another. In some embodiments, tie layers can comprise any nonpolar polymer having a polar group grafted thereon, such that the polymer is capable of covalent bonding to polar polymers such as polyamide and ethylene/vinyl alcohol copolymer. In some embodiments, tie layers can comprise at least one member selected from the group including, but not limited to, modified polyolefin, modified ethylene/vinyl acetate copolymer, and/or homogeneous ethylene/alpha-olefin copolymer. In some embodiments, tie layers can comprise at least one member selected from the group consisting of anhydride modified grafted linear low density polyethylene, anhydride grafted low density polyethylene, homogeneous ethylene/alpha-olefin copolymer, and/or anhydride grafted ethylene/vinyl acetate copolymer.

Although the majority of the above definitions are substantially as understood by those of skill in the art, one or more of the above definitions can be defined hereinabove in a manner differing from the meaning as ordinarily understood by those of skill in the art, due to the particular description herein of the presently disclosed subject matter.

### II. Package 10

II.A. Generally

As discussed hereinabove in FIGS. 1a and 1b, package 10 comprises lower sheet 15, a portion of which has been formed into pocket 20 that houses a product to be sterilized. Lidding sheet 30 is adhered to lower sheet 15 via seal 35 in the area surrounding pocket 20. Advantageously, package 10 offers the possibility of individually packaging one or more products and of being opened at the moment the sterilized product is to be used. The disclosed packages therefore allow the sterilized products to be stored under proper sterility conditions.

The presently disclosed sealed package can be reliably opened without the use of excess force. In addition, upon opening package 10, there is no formation and scattering of paper dust and the resulting opening is neat and clean. Thus, package 10 comprises very desirable easy open characteristics as well as fiber-free peeling, which are important features from the standpoint of sanitation in medical and various other settings.

II.B. Lower Sheet 15

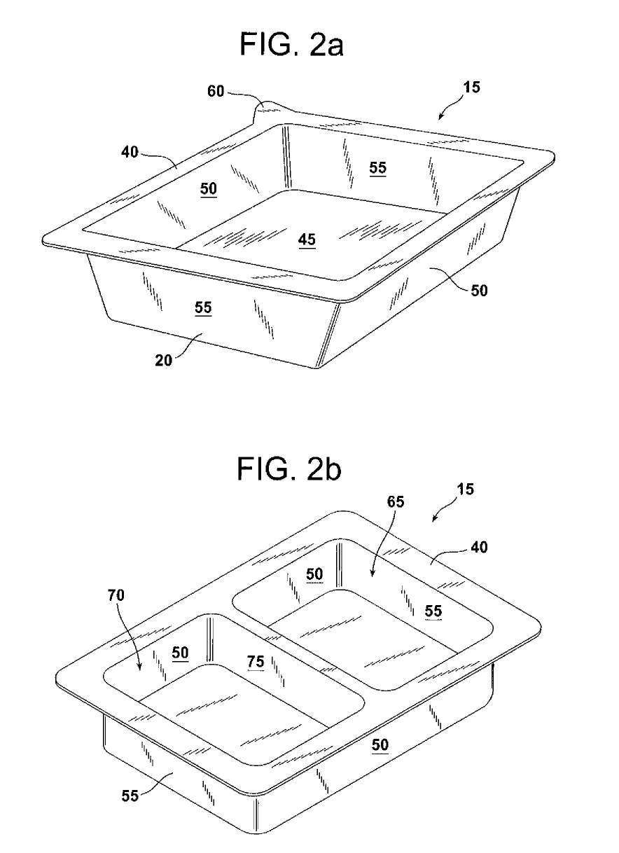

FIG. 2a illustrates one embodiment of lower sheet 15 of package 10. Particularly, lower sheet 15 includes bottom wall 45 having a pair of spaced side walls 50 and a pair of spaced end walls 55 extending upwardly therefrom. Side walls 50 and 55 are interconnected with each other and cooperate with bottom wall 45 of lower film 15 to define internal pocket 20 within which product 25 can be placed using any conventional means known in the art. Thus, in some embodiments, package 10 comprises lower sheet 15 having bottom and side walls defining an interior space with an open top.

In some embodiments, each side wall and end wall 50, 55 of lower sheet 15 can comprise a laterally extending sealing flange 40 at its upper end. Sealing flanges 40 are interconnected with each other to define a peripheral sealing surface to which lidding sheet 30 can be sealed.

Lower sheet 15 can have any desired configuration or shape, e.g., rectangular, round, oval, and the like. Similarly, flange 40 can have any desired shape or design, including a simple, substantially flat design that presents a single sealing surface as shown in the figures, or a more elaborate design that presents two or more sealing surfaces, as disclosed in U.S. Pat. Nos. 5,348,752 to Gorlich and 5,438,132 to Bray et al., the disclosures of which are incorporated herein in their entireties. In some embodiments, flange 40 can also include a peripheral lip positioned adjacent and exterior to the sealing surface to facilitate the peel of lidding sheet 30 (i.e., tab 60).

Although lower sheet can comprise only one compartment to house product 25, it is within the scope of the presently disclosed subject matter that lower sheet 15 can be formed with more than one compartments to house a plurality of products. For example, as illustrated in FIG. 2b, lower sheet 15 can comprise first and second compartments 65 and 70. In such embodiments, compartments 65 and 70 can be separated by middle wall 75 to separately house a plurality of products.

Lower sheet 15 can be constructed from any of a wide variety of materials known in the art that are impervious to bacteria and other pathogens. For example, materials suitable for constructing lower sheet 15 can include, but are not limited to, polyester, nylon, polyethylene, cellophane, polypropylene, polyvinyl acetate, saran, ethylene vinyl alcohol copolymers, vinylidene chloride copolymers (PVDC) such as vinylidene chloride vinyl chloride or vinylidene chloride methyl acrylate, or combinations of the aforementioned materials with each other or in further combination with polyethylene, ethylene vinyl acetate (EVA) copolymer, ionomer, or coextrusions involving two or more of the aforementioned polymeric materials.

For example, in some embodiments, films with a polyethylene sealant layer and alternating interior layers of ethylene/vinyl acetate copolymer and polypropylene can be used. To this end, a representative film for use in accordance with lower sheet 15 is a film having the structure: VLDPE and/or LLDPE (seal layer)/EVA/polypropylene/EVA/polypropylene/EVA/polypropylene, although a wide variety of films known in the art can be employed.

Thus, lower sheet 15 can be provided in sheet or film form and can be any of the films commonly used for the disclosed type of packaging. Accordingly, lower sheet 15 can comprise one or more barrier layers, seal layers, tie layers, abuse layers, and/or bulk layers. The polymer components used to fabricate lower sheet 15 can also comprise appropriate amounts of other additives normally included in such compositions. For example, slip agents (such as talc), antioxidants, fillers, dyes, pigments and dyes, radiation stabilizers, antistatic agents, elastomers, and the like can be added to the disclosed films. See, for example, U.S. Pat. Nos. 7,205,040 to Peiffer et al.; 7,160,378 to Eadie et al.; 7,160,604 to Ginossatis; 6,472,081 to Tsai et al.; 6,222,261 to Horn et al.; 6,221,470 to Ciacca et al.; 5,591,520 to Migliorini et al.; and 5,061,534 to Blemberg et al., the disclosures of which are hereby incorporated by reference in their entireties.

Lower sheet 15 can be constructed by any suitable process known to those of ordinary skill in the art, including (but not limited to) coextrusion, lamination, extrusion coating, and combinations thereof. See, for example, U.S. Pat. No. 6,769,227 to Mumpower, the content of which is herein incorporated by reference in its entirety.

Generally, films employed in lower sheet 15 can be multilayer or monolayer. Typically, however, the films employed will have two or more layers in order to incorporate a variety of properties, such as, for example, sealability, gas impermeability, and toughness into a single film. Thus, in some embodiments, lower sheet 15 comprises a total of from about 4 to about 20 layers; in some embodiments, from about 4 to about 12 layers; and in some embodiments, from about 5 to about 9 layers. Accordingly, the disclosed film can comprise 1, 2, 3, 4, 5, 6, 7, 8, 9, 10, 11, 12, 13, 14, 15, 16, 17, 18, 19, or 20 layers. One of ordinary skill in the art would also recognize that sheet 15 can comprise more than 20 layers, such as in embodiments wherein the sheet components comprise microlayering technology.

Lower sheet 15 can have any total thickness desired, so long as it provides the desired properties for the particular packaging operation in which it is used, e.g., optics, modulus, seal strength, and the like. Final web thicknesses can vary, depending on process, end use application, and the like. Typical thicknesses can range from about 0.1 to 20 mils; in some embodiments, about 0.3 to 15 mils; in some embodiments, about 0.5 to 10 mils; in some embodiments, about 1 to 8 mils; in some embodiments, about 1 to 4 mils; and in some embodiments, about 1 to 2 mils. For example, in some embodiments, the thickness can be from about 2 to about 12 mils.

In some embodiments, lower sheet 15 can be transparent (at least in the non-printed regions) such that product 25 is visible through the film. The term “transparent” as used herein can refer to the ability of a material to transmit incident light with negligible scattering and little absorption, enabling objects (e.g., a packaged medical device) to be seen clearly through the material under typical unaided viewing conditions (i.e., the expected use conditions of the material). The transparency of the film can be at least about any of the following values: 20%, 25%, 30%, 40%, 50%, 65%, 70%, 75%, 80%, 85%, and 95%, as measured in accordance with ASTM D1746.

In some embodiments, lower sheet 15 can comprise an easy open feature, such as tab 60, depicted in FIG. 2a. In use, one would merely peel tab 60 to separate lidding sheet 30 from lower sheet 15 to have direct access to the items contained within package 10. One of ordinary skill in the art would recognize that any of a number of suitable opening means can be included within the presently disclosed package. For example, Chevron seals, ring pull tabs, zippers, and the like can be used.

II.B. Lidding Sheet 30

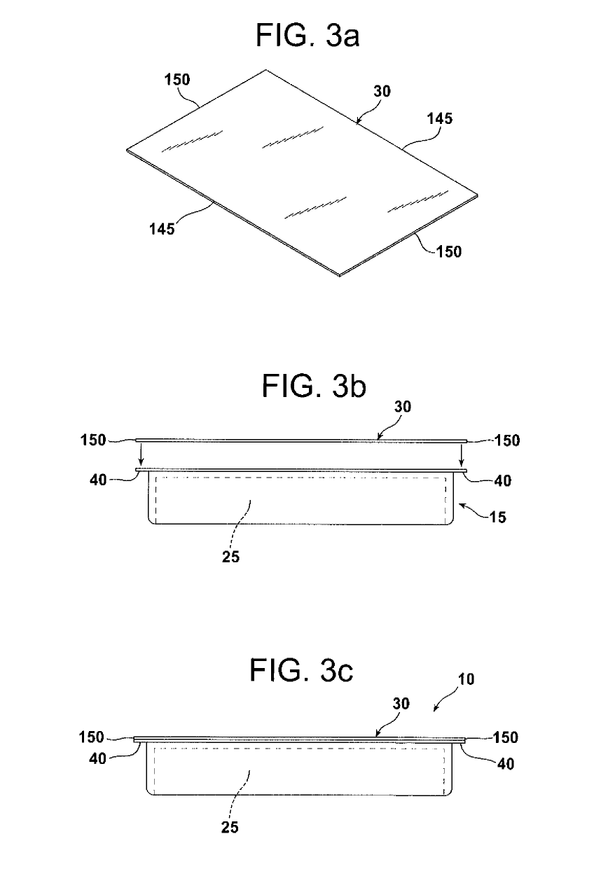

As illustrated in FIG. 3a, lidding sheet 30 comprises a pair of opposing side edges 145 and a pair of opposing end edges 150. As discussed in more detail herein below, lidding sheet 30 is sealed to lower sheet 15 around the perimeter of pocket 20 to completely contain product 25 within the interior of the formed package. Particularly, as illustrated in FIGS. 3b and 3c, edges 150 and 155 of lidding sheet 30 make contact and are sealed to flange 40 of lower sheet 15.

Essentially any gas permeable material can be used to construct lidding film 30, provided that the material is uncoated and permeable to a sterilizing gas but impermeable to microbes, bacteria, viruses, and mixtures thereof. Suitable gas permeable materials can include (but are not limited to) medical grade uncoated paper, uncoated nonwoven materials (such as Tyvek®, for example), and other similar gas permeable materials. For example, medical grade uncoated paper is manufactured by Arjo Wiggins USA (Stamford, Conn., United States of America) under the model number CP 83G.

Thus, lidding sheet 30 is highly permeable to sterilizing gas but impermeable to microorganisms to define an effective barrier to the entry of contaminating agents. The barrier property can be characterized by bacterial filtering efficiency (referred to as BFE). BFE is expressed as a percentage that represents the percentage of bacteria stopped by the sheet. In the case of package 10, it is desirable to have a BFE of at least 85%. As described herein below, after product 25 is introduced into pocket 20 of lower sheet 15 and lidding sheet 30 has been sealed to flange 40 of the lower sheet, package 10 is subjected to the action of sterilizing gases or steam.

Lidding sheet 30 also enables aseptic opening of package 10 after sterilization. Specifically, when the package is opened, because lidding sheet 30 is uncoated, no fibers or other particles become detached and deposited on the sterilized articles or in the environment surrounding the package.

In some embodiments, lidding sheet 30 can comprise printed product information such as (but not limited to) product size, type, name of manufacturer, instructions, and the like. Such printing methods are well known to those of ordinary skill in the packaging art.

II.C. Product 25

The presently disclosed packages can be used to house and sterilize a wide variety of products including (but not limited to) medical products. For example, protective garments, protective coverings, wound coverings, medical devices (such as sutures, clamps, scalpels, forceps, scissors, and the like), gloves, needles, sponges, syringes, receptacles, and the like can be used with package 10. In addition, the disclosed package is suitable for use with a variety of non-medical products, as would be apparent to those of ordinary skill in the packaging art.

### III. Methods of Making Package 10

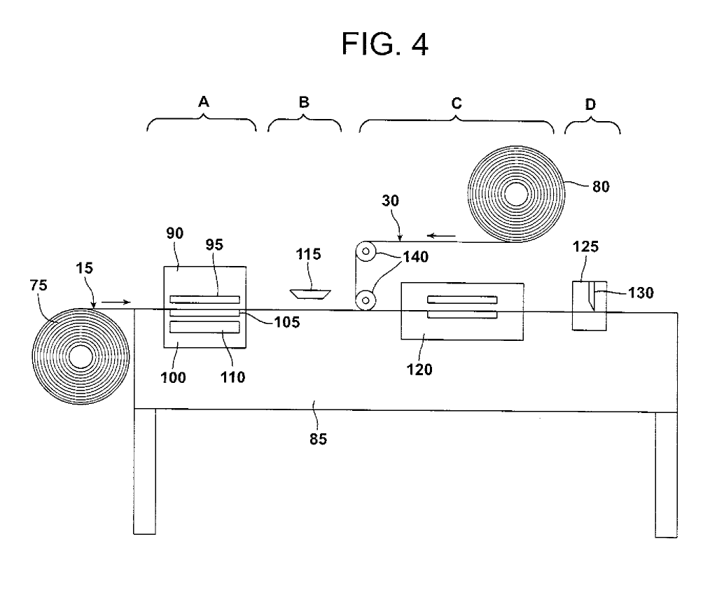

FIG. 4 illustrates one method of forming package 10. Particularly, roll 75 of lower sheet 15 is mounted on a suitable support, along with roll 80 of lidding sheet 30. Roll 75 is mounted with suitable unwind mechanisms (not shown) at the end of machine frame 85. Roll 80 is mounted with suitable unwind mechanisms (also not shown) above machine frame 85. Located at forming station A is upper half forming section 90 with heating platen 95 and lower half forming section 100 with forming tray 105. Forming tray 105 determines the shape of pocket 20 and spacers 110 determine the depth of pocket 20. The shape and quantity of heating platens 95 and forming trays 105 in the forming section will depend on the desired shape and quantity of pockets in package 10.

Lower sheet 15 is threaded into grippers (not shown) that hold the sheet on both sides throughout the process from A to D. Lower sheet 15 is drawn into the forming station A and clamped between the upper and lower halves of the forming sections 90 and 100, where vacuum and/or compressed air is used to pull and/or press the lower sheet against one or more heated platens 95 within upper forming section 90 until the sheet softens and becomes pliable. Vacuum and/or compressed air is then used to pull and/or press lower sheet 15 down into forming tray 105 within lower forming section 100 where the lower sheet is allowed to cool and form pocket 20.

The interdependent conditions that can be varied at forming station A are time, temperature, pressure and pocket depth. In some embodiments, heating platens 95 are heated to a temperature between the softening temperature and the melt temperature for the lower sheet material. In some embodiments, this temperature can be between about 80° C. and 140° C.; in some embodiments, between about 90° C. and 130° C. The air pressure applied to push lower sheet 15 against heated platens 95 can be in some embodiments between about 10 and 50 psi; in some embodiments, between about 20 and 40 psi. The time period during which lower sheet 15 is subjected to heating by platens 95 to soften the sheet and make it pliable can be in some embodiments between about 0.6 and 3.0 seconds; in some embodiments between about 1.0 and 2.5 seconds. The time period during which lower sheet 15 is allowed to cool in forming tray 105 to form pocket 20 is in some embodiments between about 0.4 and 3.0 seconds.

After pocket 20 is formed, lower sheet 15 is unclamped from forming station A and advanced to loading station B where product 25 is loaded into formed pocket 20. Particularly, at loading or filling station B, loading mechanism 115 places product 25 into the formed pocket of lower sheet 15 either automatically or by hand.

Lower sheet 15 is then advanced to sealing station C where lidding sheet 30 is guided into position from roll 80 by a pair of idler rolls 140 for correct positioning over lower sheet 15 and under sealing section 120. Sealing section 120 contains a heated die to heat-seal lidding sheet 30 to lower sheet 15 to form peel seal area 35. Sealing station C provides suitable heat and pressure to seal the desired areas of lower sheet 15 and lidding sheet 30 together under conventional conditions for heat sealing. While heat sealing has been described, other methods of adhering lidding sheet 30 to lower sheet 15 can be used, such as (but not limited to) cold seal, conventional adhesives, ultrasonic welding, and the like.

The desired seal strength can be achieved by conventional means known in the art, such as by selection of the upper and lower sheet materials, by adjustment of the thickness of the sheets, and/or by adjusting the conditions of making the seals (such as by adjusting platen temperature, dwell time, platen pressure, by using opposing heated platens, by repeating the heat sealing operation, and the like).

Thus, lidding sheet 30 and lower sheet 15 are adhered together to securely enclose product 25 within the recess of pocket 20. In some embodiments, a portion along an edge of package 10 can be left unsealed to provide an area where lidding sheet 30 can be peeled from lower sheet 15. In particular, the unsealed portion of the edge can be fanned away and then grasped and peeled away from the heat sealed areas surrounding pocket 20. After removal of lidding sheet 15, product 25 can be accessed without violating the sterile field of the product.

At cross cutter station D, sheeter 125, which contains cutting means 130, makes a cross-cut across package 10. It is recognized that razors or any suitable slitting means can be used. Finished packages 10 are then discharged from the machine.

Machines for assembly line thermoform-fill-seal packaging, such as described herein, are commercially available as Multivac 230, M855, RS200, and R7000 from Multivac, Inc. of Kansas City, Mo., United States of America.

The method and packaging apparatus described above comprises packaging product 25 within the pocket 20 in lower sheet 15 prior to being covered by lidding sheet 30 and sealed on all sides to completely enclose the product. However, it is also within the scope of the presently disclosed subject matter to seal lidding sheet 30 to lower sheet 15 on less than all sides and put product 25 into partially sealed package 10. After product 25 is placed within the package, sealing of lidding sheet 30 to lower sheet 15 is completed.

It will be apparent to those of ordinary skill in the art that the apparatus and method described above and in FIG. 4 are merely exemplary of one method of making a package and that other methods and machinery well known in the art can be utilized for fabrication of other shapes and configurations of package 10.

### IV. Methods of Using Package 10

The package made in accordance with the presently disclosed subject matter provides acceptable medical type packaging for a large variety of items. The packaging and enclosed product can be subjected to sterilization and the sterilant (such as, for example, ethylene oxide gas) will readily penetrate lidding sheet 30 to sterilize product 25 housed within pocket 20.

For example, an ethylene oxide sterilization process can be used to sterilize package 10. Those skilled in the art will appreciate that although ethylene oxide gas is a preferred sterilant gas, any sterilant gas can be used with the presently disclosed package. After package 10 has been formed as discussed herein above, it is placed into a conventional ethylene oxide sterilization unit. Prior to the start of the cycle, the sterilizer is heated to an internal temperature of about 25° C. Next, a vacuum of approximately 1.8 to 6.0 kpa is drawn on the sterilization unit. Steam is then injected to provide a source of water vapor for the product to be sterilized. Package 10 is exposed to water vapor in the sterilizer for about 60 minutes to about 90 minutes.

Following the humidification portion of the cycle, the sterilizer is pressurized to about 46 to 48 kPa by the introduction of dry nitrogen gas. When the desired pressure is reached, pure ethylene oxide is introduced into the sterilization unit until the pressure reaches about 95 kpa. The ethylene oxide sterilant gas is maintained in the sterilization unit for about 360 to about 600 minutes, although the time required to sterilize can vary depending on the type of product and the package.

The ethylene oxide sterilant gas is then evacuated from the sterilization unit and the vessel is maintained under vacuum at a pressure of approximately 0.07 kpa for approximately two hours to remove residual moisture and ethylene oxide from the sterilized package. The pressure in the sterilizer is then returned to atmospheric pressure at a temperature of about 21° C. to about 32° C. The product in package 10 is then dried by exposing the package to dry nitrogen and vacuum over a number of cycles sufficient to effectively remove residual moisture and water vapor from the product and package. The package is then removed from the sterilizer and can be stored at ambient temperatures to await use.

One of ordinary skill in the art would recognize that any conventional ethylene oxide gas process can be used that is sufficient to effectively sterilize a packaged medical device, and the presently disclosed subject matter is not limited to the sterilization method disclosed above.

It is obviously important that package 10 be reliably sealed, and that it remains sealed after sterilization such that its sterilized contents remain sterilized for the required time, generally for at least 30 days. It is also important, however, that the sealed sterilized package is reliably opened without requiring excessive force and without the risk of generating fiber “dust”.

To this end, package 10 can be opened manually by grasping lidding sheet 30 with one hand and simultaneously grasping lower sheet 15 with the other hand, and then pulling apart as depicted in FIG. 1b. The force required to pull a seal apart is called “seal strength” and can be measured in accordance with ASTM F88-94. The desired seal strength varies according to specific end user applications. In some embodiments, the force required to separate lidding sheet 30 from lower sheet 15 can be about 1.0 lb/inch; in some embodiments, between 1.0 lb/inch and 2.0 lbs/inch.

In some embodiments, to facilitate opening package 10, one or more opening means can be included. For example, as depicted in FIG. 2a, package 10 can comprise a gripping tab or other suitable means to allow for ease in gripping lidding sheet 30. In some embodiments, lidding sheet 30 can be substantially the same size as lower sheet 15, but slightly shorter at one edge to facilitate separation. One of ordinary skill in the art would appreciate that the presently disclosed subject matter includes embodiments wherein no easy open means is present in package 10.

### V. Benefits of Package 10

The disclosed package is particularly suited for the packaging of medical and other devices. Specifically, the package comprises a lower sheet, a portion of which has been formed into a pocket that houses a product to be sterilized. An uncoated lidding sheet is adhered to the lower sheet via a heat seal in the area surrounding the pocket. Advantageously, the uncoated lidding sheet is much less expensive compared to coated medical grade paper or coated Tyvek® sheets currently used in the packaging art.

Upon opening the presently disclosed package, fiber pull is essentially eliminated. That is, package 10 avoids the tendency for prior art packages to separate such that the fibers pull apart from each other during opening. Thus, the disclosed package maintains the sterility of the packaged product and the surrounding environment by eliminating fiber pull.

The presently disclosed package also allows for sterilization of the packaged product, but also provides a barrier to microorganisms. Specifically, lower sheet 15 of package 10 is impermeable to gas and microorganisms, while uncoated lidding sheet 30 is permeable to sterilizing gas and is impermeable to microorganisms. Accordingly, package 10 allows for the sterilization of a packaged product, but also maintains sterility until the package is opened.

In addition, package 10 can be reliably opened without the use of excess force, thus obviating the need to cut and/or pierce the package with a knife, scissors, or any other sharp implement.

Further, the disclosed package allows for a desired seal strength with no paper tear.

Although several advantages of the disclosed system are set forth in detail herein, the list is by no means limiting. Particularly, one of ordinary skill in the art would recognize that there can be several advantages to the disclosed package and methods that are not included herein.

### EXAMPLES

The following Examples provide illustrative embodiments. In light of the present disclosure and the general level of skill in the art, those of ordinary skill in the art will appreciate that the following Examples are intended to be exemplary only and that numerous changes, modifications, and alterations can be employed without departing from the scope of the presently disclosed subject matter.

Several film structures in accordance with the presently disclosed subject matter and comparatives are identified herein below in Tables 1 and 2.

TABLE 1
Resin Identification

| | Trade Name or | |
|---|---|---|
| Material Code | Designation | Source |
| A | AB60051LD | IMCD Italia SpA (Milan, |
| | | Italy) |
| B | Elite 5230G | Dow Chemical Company |
| | | (Midland, Michigan, |
| | | United States of America) |
| C | ADMER NF518E | Mitsui Petrochemical |
| | | Corporation (New York, |
| | | New York, United States |
| | | of America) |
| D | Grilon F 34 Natur 6030 | EMS-Chemie, Inc. |
| | | (Sumter, South Carolina, |
| | | United States of America) |
| E | SOARNOL ET3803 | Nippon Gohsei (Tokyo, |
| | | Japan) |
| F | Admer Qf300e | Mitsui Petrochemical |
| | | Corp. (New York, New |
| | | York, United States of |
| | | America) |
| G | RE216 CF | Borealis Compounds, |
| | | Inc. (Port Murray, New |
| | | Jersey, United States of |
| | | America) |
| H | 10853 | Ampacet (Tarrytown, |
| | | New York, United States |
| | | of America) |
| I | ESCORENE LD-200.48 | ExxonMobile (Fairfax, |
| | | Virginia, United States of |
| | | America) |
| J | EXCEED 4518PA | ExxonMobile (Fairfax, |
| | | Virginia, United States of |
| | | America) |
| K | PLEXAR PX3236 | LyondellBasell Industries |
| | | (Rotterdam, Netherlands) |
| L | Grivory G21 Natural | EMS-Chemie, Inc. |
| | | (Sumter, South Carolina, |
| | | United States of America) |
| M | H100QP | Honeywell International, |
| | | Inc. (Morristown, New |
| | | Jersey, United States of |
| | | America) |
| N | BYNEL 39E660 | E. I. DuPont de Nemours |
| | | and Company |
| | | (Wilmington, Delaware, |
| | | United States of America) |
| O | GRILON MB 3361 FS | EMS-Chemie, Inc. |
| | NATURAL | (Sumter, South Carolina, |
| | | United States of America) |
| P | 1080864S | Clariant Corporation |
| | | (Charlotte, North |
| | | Carolina, United States of |
| | | America) |
| Q | Ultramid B33LN 01 | BASF Corporation |
| | | (Florham Park, New |
| | | Jersey, United States of |
| | | America) |
| R | EXACT 3024 | ExxonMobile (Fairfax, |
| | | Virginia, United States of |
| | | America) |
| S | FSU 255E | A. Shulman, Inc. (Akron, |
| | | Ohio, United States of |
| | | America) |
| T | Escorene Ultra LD 721.IK | ExxonMobile (Fairfax, |
| | | Virginia, United States of |
| | | America) |
| U | Pro-fax SR257M | LyondellBasell Industries |
| | | (Rotterdam, Netherlands) |
| V | 40604 | Ampacet (Tarrytown, |
| | | New York, United States |
| | | of America) |
| W | Basell Pro-Fax PH835 | LyondellBasell Industries |
| | | (Rotterdam, Netherlands) |
| X | AMPLIFY IO 3701 | Dow Chemical Company |
| | | (Midland, Michigan, |
| | | United States of America) |
| Y | CONPOL 13B | E. I. DuPont de Nemours |
| | | and Company |
| | | (Wilmington, Delaware, |
| | | United States of America) |
| Z | TAFMER P-0480 | Mitsui Petrochemical |
| | | Corporation (New York, |
| | | New York, United States |
| | | of America) |
| AA | Ultramid B40 | BASF Corporation |
| | | (Florham Park, New |
| | | Jersey, United States of |
| | | America) |
| BB | Ultramid c40 L 01 | BASF Corporation |
| | | (Florham Park, New |
| | | Jersey, United States of |
| | | America) |
| CC | VERSIFY 3000 | Dow Chemical Company |
| | | (Midland, Michigan, |
| | | United States of America) |

A is an antiblock masterbatch with density of 1.030 g/cm³ and melt flow rate at 190° C./2.16 kg of 3.3 g/10 minutes.

B is a linear low density ethylene/octene copolymer with density of 0.916 g/cm³, a melt flow rate at 190° C./2.16 kg of 4.0 g/10 minutes, and a melting point of 122° C.

C is a maleic anhydride-modified polyethylene tie layer with density of 0.91 g/cm³ and melt flow rate at 190° C./2.16 kg of 3.1 g/10 minutes.

D is polyamide-6 with a density of 1.14 g/cm³ and melting point of 222° C.

E is ethylene/vinyl alcohol copolymer with 36.5-39.5 mole percent ethylene, density of 1.17 g/cm³, melt flow rate at 210° C./2.16 kg of 3.2 g/10 min, and melting point of 173° C.

F is a maleic anhydride-modified polypropylene tie layer with density of 0.91 g/cm³, melt flow rate at 230° C./2.16 kg of 7.0 g/10 min, and Vicat softening point of 146° C.

G is a propylene/ethylene copolymer with density of 0.905 g/cm³, melt flow rate at 230° C./2.16 kg of 11 g/10 min., melting point of 145° C., and Vicat softening point of 130° C.

H is an antiblock masterbatch with a density of 1.0 g/cm³ and melt flow rate at 190° C./2.16 kg of 1.5 g/10 min.

I is a polyethylene low density homopolymer with a density at 23° C. of 0.915 g/cm³, melt flow rate at 190° C./2.16 kg of 7.5 g/10 min., and melting point of 104° C.

J is linear low density ethylene/hexene copolymer with density at 23° C. of 0.918 g/cm³, melt flow rate at 190° C./2.16 kg of 4.5 g/10 min., and melting point of 115° C.

K is a linear low density maleic anhydride-modified polyethylene with density at 23° C. of 0.9210 g/cm³, melt flow rate at 190° C./2.16 kg of 2.0 g/10 min., melting point of 125° C., and Vicat softening point of 100° C.

L is an amorphous polyamide with density of 1.18 g/cm³, glass transition temperature of 125° C., and a refractive index of 1.58.

M is a polyamide-6 with density of 1.13 g/cm³ and melting point of 220° C.

N is maleic anhydride-modified ethylene/vinyl acetate copolymer with 11.8% vinyl acetate content, density at 23° C. of 0.9430 g/cm³, melt flow rate at 190° C./2.16 kg of 2.5 g/10 min, melting point of 95° C., and Vicat softening point of 72° C.

O is an antiblock and slip agent masterbatch with density of 1.14 g/cm³ and melting point of 220° C.

P is an antiblock and slip agent masterbatch with density of 1.2 g/cm³ and melting point of 220° C.

Q is a polyamide-6 with density of 1.14 g/cm³ and melting point of 220° C.

R is a very low density polyethylene with density of 0.9050 g/cm³, melt flow rate at 190° C./2.16 kg of 4.5 g/10 min., and melting point of 98.0° C.

S is an antiblock and slip masterbatch with density of 1.08 g/cm³ and melt flow rate at 190° C./2.16 kg of 8.0 g/10 min.

T is an ethylene/vinyl acetate copolymer with 18.5% vinyl acetate content, density at 23° C. of 0.942 g/cm³, and melt flow rate at 190° C./2.16 kg of 2.55 g/10 min.

U is a propylene/ethylene copolymer with density of 0.902 g/cm³, melt flow rate at 230° C./2.16 kg of 2.0 g/10 min., and melting point of 152° C.

V is a masterbatch slip agent with density of 0.899 g/cm³ and melt flow rate at 230° C./2.16 kg of 20.5 g/10 min.

W is a polypropylene homopolymer with density of 0.902 g/cm³ and melt flow rate at 230° C./2.16 kg of 34.0 g/10 min.

X is an ionomer with density of 0.940 g/cm³, melt flow rate at 190° C./2.16 kg of 5.2 g/10 min., melting point of 95° C., and Vicat softening point of 80° C.

Y is masterbatch antiblock with density of 1.01 g/cm³, melt flow rate at 190° C./2.16 kg of 7.5 g/10 min., and melting point of 100° C.

Z is propylene/ethylene copolymer with 20% propylene content, density of 0.87 g/cm³, melt flow rate at 230° C./2.16 kg of 1.8 g/10 min., and melting point of 41° C.

AA is a polyamide-6 with density of 1.13 g/cm³ and melting point of 220° C.

BB is polyamide 6/66 with density at 23° C. of 1.12 g/cm³, melting point of 190° C., and relative viscosity of 4.0 g/cm³.

CC is a propylene/ethylene copolymer with 5.2% ethylene content, density of 0.891 g/cm³, melt flow rate at 230° C./2.16 kg of 8.0 g/10 min., and melting point of 112° C.

TABLE 2
Film Identification

| Film ID | Layer | Formulation | | Volume % | Mils |
|---|---|---|---|---|---|
| Film 1 | 1 | 2% | A | 10.0 | 0.69 |
| | | 98% | B | | |
| | 2 | 100% | B | 17.0 | 1.17 |
| | 3 | 100% | C | 8.0 | 0.55 |
| | 4 | 100% | D | 10.0 | 0.69 |
| | 5 | 100% | E | 9.0 | 0.62 |
| | 6 | | | 10.0 | 0.69 |
| | 7 | 100% | F | 6.0 | 0.41 |
| | 8 | 100% | G | 30.0 | 2.07 |
| Film 2 | 1 | 2% | H | 8.0 | 0.2 |
| | | 10% | I | | |
| | | 88% | J | | |
| | 2 | 10% | I | 25.0 | 0.63 |
| | | 90% | J | | |
| | 3 | 100% | K | 8.0 | 0.20 |
| | 4 | 20% | L | 6.5 | 0.16 |
| | | 80% | M | | |
| | 5 | 100% | K | 8.0 | 0.20 |
| | 6 | 20% | L | 6.5 | 0.16 |
| | | 80% | M | | |
| | 7 | 100% | N | 25.0 | 0.63 |
| | 8 | 2% | O | 13.0 | 0.33 |
| | | 2% | P | | |
| | | 96% | Q | | |
| Film 3 | 1 | 6% | S | 12.0 | 0.42 |
| | | 94% | R | | |
| | 2 | 10% | I | 21.0 | 0.74 |
| | | 90% | J | | |
| | 3 | 100% | T | 7.0 | 0.25 |
| | 4 | 100% | U | 13.0 | 0.46 |
| | 5 | 100% | T | 7.0 | 0.25 |
| | 6 | 100% | U | 13.0 | 0.46 |
| | 7 | 100% | T | 15.0 | 0.53 |
| | 8 | 2% | V | 12.0 | 0.42 |
| | | 98% | W | | |
| Film 4 | 1 | 6% | S | 15.0 | 0.38 |
| | | 94% | R | | |
| | 2 | 10% | I | 20.0 | 0.50 |
| | | 90% | J | | |
| | 3 | 100% | K | 8.0 | 0.20 |
| | 4 | 20% | L | 6.5 | 0.16 |
| | | 80% | M | | |
| | 5 | 100% | E | 8.0 | 0.20 |
| | 6 | 20% | L | 6.5 | 0.16 |
| | | 80% | M | | |
| | 7 | 100% | N | 10.0 | 0.25 |
| | 8 | 100% | N | 13.0 | 0.33 |
| | 9 | 2% | O | 13.0 | 0.33 |
| | | 2% | P | | |
| | | 96% | Q | | |
| Film 5 | 1 | 2% | Y | 10.0 | 0.30 |
| | | 98% | X | | |
| | 2 | 10% | I | 15.0 | 0.45 |
| | | 35% | Z | | |
| | | 55% | J | | |
| | 3 | 100% | K | 8.0 | 0.24 |
| | 4 | 70% | AA | 13.0 | 0.39 |
| | | 30% | BB | | |
| | 5 | 100% | E | 10.0 | 0.30 |
| | 6 | 30% | BB | 13.0 | 0.39 |
| | | 70% | AA | | |
| | 7 | 100% | K | 7.0 | 0.21 |
| | 8 | 100% | CC | 16.0 | 0.48 |
| | 9 | 100% | W | 8.0 | 0.24 |

### Example 1

### Manufacture of Package 1

Film 1, with the composition and construction shown in Table 2, was formed by coextrusion. Film 1 was loaded onto a Multivac Model R230 packaging machine (available from Multivac, Wolfertschwenden, Germany) with the sealant layer side facing upwards as conveyed. The film was then heated and thermoformed with the assistance of a vacuum to form a pocket. The packages were empty.

A roll of uncoated medical grade paper (CP 83G available from Arjo Wiggins USA, Stamford, Conn., United States of America) was loaded onto the Multivac Model R230 machine. The machine was then indexed forward to convey the loaded support member to the vacuum packaging station of the Multivac R230. At this station, the uncoated paper was brought into contact with Film 1. Heat and pressure were applied to hermetically heat seal Film 1 and the uncoated paper together along the perimeter of the pocket. 6 replicates of package 1 (labeled as Packages 1a-1f) were made with varying heat seal times and temperatures, as set forth in Table 3 below.

TABLE 3
Time/Temperature Variations for Packages 1a-1f

| Package | 1a | 1b | 1c | 1d | 1e | 1f |
|---|---|---|---|---|---|---|
| Temp (° C.) | 120 | 125 | 130 | 130 | 135 | 140 |
| Time (sec) | 2.5 | 1.5 | 1.0 | 2.5 | 1.0 | 1.5 |

### Example 2

### Manufacture of Package 2

Film 2 was thermoformed and sealed to uncoated medical grade paper (CP83G) as in Example 1 to produce Package 2. 4 replicates of Package 2 (labeled as Packages 2a-2d) were made with varying heat seal times and temperatures, as set forth in Table 4 below.

TABLE 4
Time/Temperature Variations for Packages 2a-2d

| Package No. | 2a | 2b | 2c | 2d |
|---|---|---|---|---|
| Temp (° C.) | 120 | 125 | 130 | 130 |
| Time (sec) | 2.5 | 2.5 | 1.0 | 2.5 |

### Example 3

### Manufacture of Package 3

Film 3 was thermoformed and sealed to uncoated medical grade paper (CP83G) as in Example 1 to produce Package 3. 5 replicates of Package 3 (labeled as Packages 3a-3e) were made with varying heat seal times and temperatures, as set forth in Table 5 below.

TABLE 5
Time/Temperature Variations for Packages 3a-3e

| Package No. | 3a | 3b | 3c | 3d | 3e |
|---|---|---|---|---|---|
| Temp (° C.) | 115 | 120 | 120 | 125 | 130 |
| Time (sec) | 1.5 | 1.5 | 2.5 | 2.5 | 1.0 |

### Example 4

### Manufacture of Package 4

Film 4 was thermoformed and sealed to uncoated medical grade paper (CP83G) as in Example 1 to produce Package 4. 4 replicates of Package 4 (labeled as Packages 4a-4d) were made with varying heat seal times and temperatures, as set forth in Table 6 below.

TABLE 6
Time/Temperature Variations for Packages 4a-4d

| Package No. | 4a | 4b | 4c | 4d |
|---|---|---|---|---|
| Temp (° C.) | 115 | 120 | 120 | 130 |
| Time (sec) | 1.5 | 1.5 | 2.5 | 1.0 |

### Example 5

### Manufacture of Packages 5a-5e

Film 5 was thermoformed and sealed to uncoated medical grade paper (CP83G) as in Example 1 to produce Package 5. 5 replicates of Package 5 (labeled as Packages 5a-5e) were made with varying heat seal times and temperatures, as set forth in Table 7 below.

TABLE 7
Time/Temperature Variations for Package 5

| Package No. | 5a | 5b | 5c | 5d | 5e |
|---|---|---|---|---|---|
| Temp (° C.) | 115 | 115 | 120 | 120 | 130 |
| Time (sec) | 1.0 | 1.5 | 1.5 | 2.5 | 1.0 |

### Example 6

### Seal Strength Testing of Packages 1a-1f

Seal strength testing, also known as “peel testing”, was performed on the replicates of Package 1. Seal strength testing measures the strength of seals within the packages and can be used to determine consistency within the seal, as well as to evaluate the opening force of the package. Seal strength is a qualitative measure for use in process validation, process control, and capability. Seal strength is not only relevant to opening force and package integrity, but also to measuring the packaging processes' ability to produce consistent seals.

On the Instron 5543, using a standard seal strength test method, the seal between the package edge and the peel seal area was tested for each replicate. In the Instron tests performed, an inch-wide cut was taken perpendicular to the peel seal area, leaving a flap attached to the bottom and top material sealed together. Each flap was inserted into a jaw on the Instron and a pull cycle started. The resulting seal strengths were measured in pounds/inch.

For Packages 1a-1f, 9 seals were tested from each package.

The seal strength data for Packages 1a-1f is presented below in Table 8.

From the data, Package 1d is the only package where the average seal strength was in the optimal range of 1-2 lbs/in.

TABLE 8
Seal Strength Testing of Packages 1a-1f

| Pckg No. | 1a | 1b | 1c | 1d | 1e | 1f |
|---|---|---|---|---|---|---|
| 1 | 0.658 | 0.655 | 0.454 | 1.327 | 0.619 | 0.687 |
| 2 | 0.724 | 0.672 | 0.403 | 1.417 | 0.593 | 0.635 |
| 3 | 0.572 | 0.985 | 0.588 | 1.489 | 0.662 | 0.464 |
| 4 | 0.551 | 0.885 | 0.523 | 1.517 | 0.394 | 0.379 |
| 5 | 0.604 | 0.636 | 0.553 | 1.435 | 0.450 | 0.752 |
| 6 | 0.765 | 0.522 | 0.571 | 1.272 | 0.370 | 0.772 |
| 7 | 0.457 | 0.775 | 0.537 | 1.300 | 0.419 | 0.749 |
| 8 | 0.506 | 1.06 | 0.549 | 1.341 | 0.596 | 0.524 |
| 9 | 0.503 | 1.049 | 0.464 | 1.349 | 0.629 | 0.460 |
| average | 0.5933 | 0.8043 | 0.5158 | 1.383 | 0.5258 | 0.6024 |
| Std. dev. | 0.10456 | 0.19783 | 0.06172 | 0.8538 | 0.11518 | 0.14847 |

### Example 7

### Seal Strength Testing of Packages 2a-2d

Seal strength testing was performed for Packages 2a-2d as in Example 6. For Packages 2a-2d, 9 seals were tested from each package and the data reported in Table 9 below.

From the data, Package 2b is the only package where the average seal strength was in the optimal range of 1-2 lbs/in.

TABLE 9
Seal Strength Testing of Packages 2a-2d

| Pckg No. | 2a | 2b | 2c | 2d |
|---|---|---|---|---|
| 1 | 0.456 | 1.259 | 0.440 | 1.606 |
| 2 | 0.629 | 1.189 | 0.405 | 0.902 |
| 3 | 0.584 | 0.938 | 0.863 | 1.024 |
| 4 | 0.387 | 1.527 | 0.584 | 0.843 |
| 5 | 0.742 | 0.677 | 0.983 | 0.781 |
| 6 | 0.394 | 1.133 | 0.494 | 0.957 |
| 7 | 0.338 | 1.531 | 0.873 | 1.046 |
| 8 | 0.381 | 1.443 | 0.868 | 0.830 |
| 9 | 0.424 | 0.602 | 0.398 | 0.815 |
| average | 0.4817 | 1.1443 | 0.6559 | 0.9782 |
| Std. dev. | 0.13755 | 0.3456 | 0.23648 | 0.25319 |

### Example 8

### Seal Strength Testing of Packages 3a-3e

Seal strength testing was performed for packages 3a-3e as in Example 6. For Packages 3a-3e, 9 seals were tested from each package and the data reported in Table 10 below.

From the data, Packages 3b, 3c, 3d, and 3e were in the optimal range of 1-2 lbs/in.

TABLE 10
Seal Strength Testing of Packages 3a-3e

| Pckg No. | 3a | 3b | 3c | 3d | 3e |
|---|---|---|---|---|---|
| 1 | 0.601 | 1.389 | 1.093 | 1.770 | 0.633 |
| 2 | 0.567 | 1.609 | 1.342 | 1.203 | 1.347 |
| 3 | 0.602 | 1.108 | 1.522 | 1.636 | 1.211 |
| 4 | 0.527 | 0.693 | 1.495 | 1.670 | 1.502 |
| 5 | .563 | 1.271 | 1.004 | 1.556 | 0.853 |
| 6 | 0.457 | 0.878 | 1.046 | 1.700 | 0.844 |
| 7 | 0.694 | 1.377 | 1.614 | 1.291 | 0.679 |
| 8 | 0.584 | 1.641 | 0.812 | 1.616 | 1.265 |
| 9 | 0.599 | 0.906 | 1.019 | 0.981 | 0.980 |
| average | 0.5771 | 1.208 | 1.2163 | 1.4914 | 1.0349 |
| Std. dev. | 0.06385 | 0.33334 | 0.28212 | 0.26867 | 0.30828 |

### Example 9

### Seal Strength Testing of Packages 4a-4d

Seal strength testing was performed for Packages 4a-4d as in Example 6. For Packages 4a-4d, 9 seals were tested from each package and the data reported in Table 11 below.

From the data, only Packages 4b and 4c were in the optimal range of 1-2 lbs/in.

TABLE 11
Seal Strength Testing of Packages 4a-4d

| Pckg No. | 4a | 4b | 4c | 4d |
|---|---|---|---|---|
| 1 | 0.499 | 1.241 | 1.183 | 0.801 |
| 2 | 0.477 | 1.198 | 0.968 | 1.115 |
| 3 | 0.358 | 1.023 | 1.222 | 1.055 |
| 4 | 0.704 | 1.023 | 1.263 | 0.768 |
| 5 | 0.542 | 1.178 | 1.158 | 1.041 |
| 6 | 0.412 | 1.139 | 1.212 | 0.735 |
| 7 | 0.793 | 1.048 | 1.130 | 0.968 |
| 8 | 0.498 | 1.039 | 0.971 | 1.116 |
| 9 | 0.708 | 1.287 | 1.211 | 0.950 |
| average | 0.5546 | 1.1307 | 1.1464 | 0.9499 |
| Std. dev. | 0.14754 | 0.10117 | 0.10731 | 0.14831 |

### Example 10

### Seal Strength Testing of Packages 5a-5e

Seal strength testing was performed for Packages 5a-5e as in Example 6.

For Packages 5a-5e, 9 seals were tested from each package and the data reported in Table 12 below.

From the data, no packages were in the optimal range of 1-2 lbs/in.

TABLE 12
Seal Strength Testing of Package 5a-5e

| Pckg No. | 5a | 5b | 5c | 5d | 5e |
|---|---|---|---|---|---|
| 1 | 0.487 | 0.509 | 0.520 | 0.657 | 0.539 |
| 2 | 0.477 | 0.399 | 0.438 | 0.538 | 0.548 |
| 3 | 0.394 | 0.387 | 0.619 | 0.676 | 0.452 |
| 4 | 0.464 | 0.440 | 0.511 | 0.629 | 0.573 |
| 5 | 0.463 | 0.441 | 0.510 | 0.497 | 0.604 |
| 6 | 0.482 | 0.492 | 0.417 | 0.628 | 0.384 |
| 7 | 0.422 | 0.487 | 0.482 | 0.670 | 0.500 |
| 8 | 0.474 | 0.436 | 0.514 | 0.579 | 0.577 |
| 9 | 0.406 | 0.496 | 0.494 | 0.473 | 0.493 |
| average | 0.4521 | 0.4541 | 0.5506 | 0.5941 | 0.5189 |
| Std. dev. | 0.03514 | 0.04405 | 0.05708 | 0.07604 | 0.06937 |

### Example 11

### Peel Characteristics of Packages 1a-1f

Packages 1a-1f were manually opened by peeling the uncoated paper layer from the lower layer. The amount of paper tearing was then observed.

Significant tearing of the uncoated paper layer was observed in all of packages 1a-1f.

### Example 12

### Peel Characteristics of Packages 2a-2d

Packages 2a-2d were observed for paper tearing as in Example 11.

Significant tearing of the uncoated paper layer was observed in Packages 2b and 2d.

### Example 13

### Peel Characteristics of Packages 3a-3e

Packages 3a-3e were observed for paper tearing as in Example 11.

No tearing was observed in any of the packages, and the packages peeled open smoothly.

### Example 14

### Peel Characteristics of Packages 4a-4d

Packages 4a-4d were observed for paper tearing as in Example 11.

Tearing was observed in Packages 4b, 4c, and 4d.

### Example 15

### Peel Characteristics of Packages 5a-5e

Packages 5a-5e were observed for paper tearing as in Example 11.

No tearing was observed in any of the packages.

### CONCLUSIONS

Packages 3a-3e were the most favorable of all packages tested. Specifically, Packages 3a-3e exhibited the optimal seal strength and favorable (none or no) tearing conditions. In addition, packages 3a-3e exhibited smooth, easy and clean peel characteristics.

What is claimed is:

1. A microbial resistant package comprising:
a. a gas-impermeable polymeric material comprising a cavity formed therein for receiving a product, wherein said material has a flange surrounding said cavity;
b. a product deposited in said cavity; and
c. an uncoated gas permeable material bonded to the flange of said polymeric material.

2. The package of claim 1, wherein said gas-impermeable polymeric material comprises polyester, nylon, polyethylene, cellophane, polypropylene, polyvinyl acetate, saran, ethylene vinyl alcohol copolymers, vinylidene chloride copolymer, or combinations thereof.

3. The package of claim 1, wherein said gas-impermeable polymeric material comprises a film comprising:
a. a polyethylene sealant layer;
b. at least one alternating interior layer comprising:
i. ethylene/vinyl acetate copolymer; and
ii. polypropylene.

4. The package of claim 1, wherein said product comprises a medical product.

5. The package of claim 1, wherein said uncoated gas permeable material comprises uncoated medical grade paper, uncoated nonwoven materials, or combinations thereof.

6. A method of eliminating fiber pull from a package, said method comprising:
a. providing a package, said package comprising:
i. a gas-impermeable polymeric material comprising a cavity formed therein for receiving a product, wherein said material has a flange surrounding said cavity;
ii. a product deposited in said cavity; and
iii. an uncoated gas-permeable material bonded to the flange of said polymeric material;
b. opening said package by removing said uncoated gas-permeable material,
wherein said opening produces no fiber pull.

7. The method of claim 6, wherein said gas-impermeable polymeric material comprises polyester, nylon, polyethylene, cellophane, polypropylene, polyvinyl acetate, saran, ethylene vinyl alcohol copolymers, vinylidene chloride copolymer, or combinations thereof.

8. The method of claim 6, wherein said gas-impermeable polymeric material comprises a film comprising:
a. a polyethylene sealant layer;
b. at least one alternating interior layer comprising:
i. ethylene/vinyl acetate copolymer; and
ii. polypropylene.

9. The method of claim 6, wherein said product comprises a medical product.

10. The method of claim 6, wherein said uncoated gas permeable material comprises uncoated medical grade paper, uncoated nonwoven materials, or combinations thereof.

11. A gas impermeable polymeric film suitable for use in a package in combination with an uncoated, gas permeable material, said film comprising:
a. a polyethylene sealant layer;
b. at least one alternating interior layer comprising:
i. ethylene/vinyl acetate copolymer; and
ii. polypropylene;
wherein said film is characterized by an easy-to-open and fiber-free peel.

12. The film of claim 1, wherein said uncoated gas permeable material comprises uncoated medical grade paper, uncoated nonwoven materials, or combinations thereof.
